(19) United States
(12) Patent Application Publication
(10) Pub. No.: US 2023/0152307 A1
(43) Pub. Date: May 18, 2023

(54) NANOSCREEN AND METHOD OF REGULATING STEM CELL ADHESION AND DIFFERENTIATION USING THE SAME

(71) Applicants: Korea University Research and Business Foundation, Seoul (KR); Industry-University Cooperation Foundation Hanyang University ERICA Campus, Ansan (KR)

(72) Inventors: Heemin KANG, Seoul (KR); Do-Kyoon KIM, Ansan-si (KR); Hyun-Shik HONG, Seoul (KR); Sun-Hong MIN, Suwon-si (KR)

(73) Assignees: Korea University Research and Business Foundation, Seoul (KR); Industry-University Cooperation Foundation Hanyang University ERICA Campus, Ansan (KR)

(21) Appl. No.: 17/963,578

(22) Filed: Oct. 11, 2022

(30) Foreign Application Priority Data
Nov. 16, 2021 (KR) 10-2021-0157179

(51) Int. Cl. G01N 33/543; C12N 5/0775; C12N 13/00; A61K 47/69; G01N 33/553

(52) U.S. Cl. CPC G01N 33/54346; C12N 5/0662; C12N 13/00; A61K 47/6923; G01N 33/553; C12N 2529/00; B82Y 5/00

(57) ABSTRACT

The present invention relates to a nanoscreen for regulating stem cell adhesion and differentiation. Moreover, the present invention relates to a method of regulating stem cell adhesion and differentiation using the nanoscreen. According to the nanoscreen of the present invention and the method of regulating stem cell adhesion and differentiation using the same, it is possible to efficiently regulate stem cell adhesion and differentiation by applying a magnetic field to the nanoscreen.

### CROSS-REFERENCE TO RELATED APPLICATIONS

This application claims priority to and the benefit of Korean Patent Application No. 10-2021-0157179 filed in the Korean Intellectual Property Office on Nov. 16, 2021, the entire contents of which are incorporated herein by reference.

### BACKGROUND OF THE INVENTION

### Field of the Invention

The present invention relates to a magnetic nanoscreen, and more particularly, to a magnetic nanoscreen and a method of regulating stem cell adhesion and differentiation using the same.

### Description of the Prior Art

Physical screens, which occur in the extracellular matrix (ECM), separate various tissue compartments to help modulate homeostasis and tissue regeneration by controlling biomolecular transport and cellular infiltration. Certain tissues can act as physical screens to modulate tissue repair mechanisms that involve the interactions of diverse cells. However, ECM-mimicking artificial materials that can dispersively and dynamically control bioactive surfaces are rare.

Integrin dynamically forms links with the bioactive-ligand-displaying-ECM, of which the RGD ligands mediate focal adhesion and intracellular mechanotransduction of cells. Remote manipulation of unscreening the ligands by light or magnetic fields can dynamically modulate cell adhesion. Conventionally, light such as ultraviolet (UV), visible, and near infrared (NIR) light has been used for photochemical manipulation of screening and unscreening of the ligands. For example, UV light has been applied to chemically cleave photosensitive polyethylene glycol-based brushes to unscreen ligand-grafted gold nanoparticles for facilitating cell adhesion. Using photoisomers such as azobenzene derivatives, screening and unscreening of the ligands via self-assembled brushes can be manipulated by illuminating UV light and visible light or single wavelengths having the ability to stimulate intracellular mechanotransduction. However, manipulation of screening and unscreening of the ligands by light in vivo has rarely been reported.

In addition, magnetic field can easily penetrate tissues in vivo to enable noninvasive control of physical screens. For example, cell adhesion can be remotely controlled by controlling screening and unscreening of the ligands through modulation of nanoparticles having magnetic properties.

### PRIOR ART DOCUMENTS

### Patent Documents

Korean Patent No. 10-1918817

### SUMMARY OF THE INVENTION

An object of the present invention is to provide a magnetic nanoscreen for regulating stem cell adhesion and differentiation.

Another object of the present invention is to provide a method of regulating stem cell adhesion and differentiation using a magnetic nanoscreen.

According to one aspect of the present invention, embodiments of the present invention include a nanoscreen for regulating stem cell adhesion and differentiation.

The nanoscreen may comprise: magnetic screens each comprising an aggregate of one or more magnetic particle units; a linker connected to one side of each of the magnetic screens;

and a substrate connected to the magnetic screens via the linkers, wherein the substrate comprises ligands to which stem cells adhere.

The average diameter of the magnetic screens may include any one or more of a first average diameter, a second average diameter, and a third average diameter, wherein the first average diameter is 150 to 250 nm, the second average diameter is 450 to 580 nm, and the third average diameter is 650 to 900 nm.

The surface of each magnetic screen, which faces the substrate, may be spaced apart from each ligand present on the substrate by a distance of a nanogap, and the nanogap may be reversibly changed by application of a magnetic field.

The average diameter of the magnetic screens may include the first average diameter, and the stem cell adhesion and differentiation may be facilitated by elongating the linker and increasing the nanogap, through pulling of the magnetic screens in a direction away from the substrate by application of the magnetic field.

The average diameter of the magnetic screens may include the third average diameter, the stem cell adhesion and differentiation may be inhibited by compressing the linker and reducing the nanogap, through pulling of the magnetic screens in a direction toward the substrate by application of the magnetic field.

The linker may comprise: a polyethylene glycol (PEG) portion; a first bonding portion which forms a chemical bond with the magnetic screen; and a second bonding portion which forms a chemical bond with the substrate.

The magnetic screens may include a carboxylate group (—COO⁻), the first bonding portion may include any one of an amino group (—NH₂) and a thiol group (—SH) and form a chemical bond with the carboxylate group of the magnetic screen, and the second bonding portion may include any one of a maleimide group and an alkenyl group (—C═C—) and form a chemical bond with a thiol group (—SH) provided on the substrate.

The linker may have a structure of the following Formula 1:

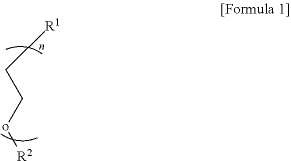

wherein R¹ is any one of an amino group (—NH₂) and a thiol group (—SH), and R² is any one of a maleimide group and an alkenyl group (—C═C—).

n in Formula 1 above may be 30 to 5,000.

The linker may have a length of 10 nm to 1 pm.

The ligands provided on the substrate may be bound to the surfaces of gold nanoparticles bound to the substrate.

The gold nanoparticles may be provided on the substrate by chemical bonding with a portion of the thiol groups (—SH) provided on the substrate, the ligands may be bound to the gold nanoparticles, and the linkers may be connected to the substrate by chemical bonding with the other portion of the thiol groups (—SH) provided on the substrate.

The gold nanoparticles may cover 0.001% to 10% of the area of the substrate.

68 to 80% of the area of the substrate may be covered by the magnetic screens.

The nanoscreen may be prepared by: forming aggregates of one or more magnetic particle units; forming a carboxylate group on the surfaces of the aggregates to form magnetic screens; binding each of the magnetic screens to one end of each linker by stirring the magnetic screens and the linkers; chemically binding the other end of each linker to thiol groups present on a substrate on which thiol groups and ligands are present; and deactivating thiol groups on the substrate, which remain unbound to the linkers.

The substrate may comprise a glass substrate, and thiol groups and ligands provided on at least one surface of the glass substrate, the thiol groups may be provided by thiolating the glass substrate, at least a portion of the thiol groups may be bound to gold nanoparticles, and the ligands may be bound to the gold nanoparticles bound to the thiol groups.

Another embodiment of the present invention includes a method of regulating stem cell adhesion and differentiation using the nanoscreen.

The method of regulating stem cell adhesion and differentiation may comprise regulating stem cell adhesion and differentiation by applying a magnetic field to the nanoscreen having the above-described characteristics.

The magnetic field may be applied from outside the body to remotely control the nanoscreen in the body.

The magnetic field may have a strength of 100 mT to 500 mT.

The regulating method may comprise facilitating stem cell adhesion and differentiation by elongating the linker through pulling of the magnetic screens in a direction away from the substrate by the magnetic field.

The regulating method may comprise inhibiting stem cell adhesion and differentiation by compressing the linker through pulling of the magnetic screens in a direction toward the substrate by the magnetic field.

According to the present invention, it is possible to provide a magnetic nanoscreen capable of regulating stem cell adhesion and differentiation.

In addition, according the present invention, it is possible to regulate stem cell adhesion and differentiation using the magnetic nanoscreen.

### BRIEF DESCRIPTION OF THE DRAWINGS

a and b of FIG. 1 schematically show a nanoscreen according to one embodiment of the present invention and a process of regulating stem cell adhesion and differentiation using the nanoscreen.

FIG. 2 schematically shows a process of preparing a magnetic screen according to one embodiment of the present invention at a test tube scale.

FIG. 3 schematically shows a process of preparing a nanoscreen according to one embodiment of the present invention.

a to g of FIGS. 4 and 5 show the results of analyzing the characteristics of a magnetic screen according to one embodiment of the present invention.

FIGS. 6 to 8 show the results of analyzing the shape of a nanoscreen according to one embodiment of the present invention.

a and b of FIG. 9 show the results of testing whether a nanogap is changed by applying a magnetic field to the nanoscreen according to one embodiment of the present invention.

a to c of FIG. 10 show the results of examining the degree of stem cell adhesion depending on to the size of the nanogap in the nanoscreen according to one embodiment of the present invention.

a and b of FIG. 11; FIG. 12; a and b of FIG. 13; and a and b of FIG. 14 show the results of examining the effect of a magnetic screen and a ligand on a method for regulating stem cell adhesion and differentiation according to one embodiment of the present invention.

a and b of FIG. 15; a and b of FIG. 16; a to c of FIG. 17; a and b of FIG. 18; FIG. 19; FIG. 20; and a and b of FIG. 21 show the results of examining whether stem cell adhesion and differentiation is regulated depending on a change in the size of the nanogap by applying a magnetic field to the nanoscreen according to one embodiment of the present invention.

a and b of FIG. 22 and a and b of FIG. 23 show the results of examining the in vivo stability of the nanoscreen according to one embodiment of the present invention and conducting an experiment on the regulation of stem cell adhesion and differentiation in vivo.

### DETAILED DESCRIPTION OF THE INVENTION

The details of other embodiments are included in the detailed description and the accompanying drawings.

The advantages and features of the present invention, and the way of attaining them, will become apparent with reference to the embodiments described below in conjunction with the accompanying drawings. However, the present invention is not limited to the embodiments disclosed below and may be embodied in a variety of different forms. Since all numbers, values and/or expressions referring to quantities of components, reaction conditions, etc., used in the present specification, are subject to the various uncertainties of measurement encountered in obtaining such values, unless otherwise indicated, all are to be understood as modified in all instances by the term “about.” Where a numerical range is disclosed herein, such a range is continuous, inclusive of both the minimum and maximum values of the range as well as every value between such minimum and maximum values, unless otherwise indicated. Still further, where such a range refers to integers, every integer between the minimum and maximum values of such a range is included, unless otherwise indicated.

In the present specification, where a range is stated for a parameter, it will be understood that the parameter includes all values within the stated range, inclusive of the stated endpoints of the range. For example, a range of 5 to 10 will be understood to include the values 5, 6, 7, 8, 9, and 10, as well as any sub-range such as 6 to 10, 7 to 10, 6 to 9, and 7 to 9, and also include any value and range between the integers which are reasonable in the context of the range stated, such as 5.5, 6.5, 7.5, 5.5 to 8.5 and 6.5 to 9. For example, a range of “10% to 30%” will be understood to include the values 10%, 11%, 12%, 13%, etc., and all integers up to and including 30%, as well as any sub-range such as 10% to 15%, 12% to 18%, 20% to 30%, etc., and also include any value between the integers which are reasonable in the context of the range stated, such as 10.5%, 15.5%, 25.5%, etc.

The present invention includes a nanoscreen for regulating stem cell adhesion and differentiation according to one embodiment and a method of regulating stem cell adhesion and differentiation using the nanoscreen.

a and b of FIG. 1 schematically show a nanoscreen according to one embodiment of the present invention and a process of regulating stem cell adhesion and differentiation using the nanoscreen. a of FIG. 1 is a schematic view showing inhibiting stem cell adhesion and differentiation using a nanoscreen comprising magnetic screens having a third average diameter. In addition, b of FIG. 1 is a schematic view showing facilitating stem cell adhesion and differentiation using a nanoscreen comprising magnetic screens having a first average diameter.

The nanoscreen according to one embodiment may comprise: magnetic screens each comprising an aggregate of one or more magnetic particle units; a linker connected to one side of each of the magnetic screens; and a substrate connected to the magnetic screens via the linkers, wherein the substrate comprises ligands to which stem cells adhere.

The average diameter of the magnetic screens may include any one or more of a first average diameter, a second average diameter, and a third average diameter. The first average diameter may be 150 to 250 nm, the second average diameter may be 450 to 580 nm, and the third average diameter may be 650 to 900 nm. Preferably, the first average diameter may be 170 nm to 230 nm, the second average diameter may be 470 nm to 530 nm, and the third average diameter may be 670 nm to 830 nm. This average diameter of the magnetic screens may correspond to an optimal size for regulating stem cell adhesion and differentiation using the nanoscreen.

Each of the magnetic screens may comprise an aggregate of one or more magnetic units. In addition, the magnetic screens may include a carboxylate group (—COOH⁻) that may be bound to the linker via a chemical bond.

The magnetic screen has a slightly polyhedral shape as shown in a and b of FIG. 1, but it may be formed in an almost spherical shape so that the inner angle formed by each face is large and the magnetic screen has a sufficiently large number of faces so to be close to a spherical shape. In the process of regulating stem cell adhesion and differentiation using such a nearly spherical nanoscreen, the magnetic screens may inhibit stem cell by preventing integrins in stem cells from binding to the ligands present under the magnetic screens.

The linker may comprise: a polyethylene glycol (PEG) portion; a first bonding portion which forms a chemical bond with the magnetic screen; and a second bonding portion forming a chemical bond with the substrate.

The polyethylene glycol portion may be composed of a polyethylene glycol and may be in the form of a long-chain. The polyethylene glycol portion may be elongated or compressed depending on the direction in which the magnetic field is applied to the nanoscreen, compared to when no magnetic field is applied. Due to the nature of the polyethylene glycol portion, the linker may be elastic. Thus, in the process of regulating stem cell adhesion and differentiation using the nanoscreen, the polyethylene glycol portion may be elongated or compressed depending on the direction of application of the magnetic field, thereby facilitating or inhibiting stem cell adhesion and differentiation. Due to the nature of the polyethylene glycol portion, the linker may be elastic. The weight-average molecular weight (Mw) of the polyethylene glycol portion may be 1,300 Da to 132,000 Da, preferably 3,900 Da to 132,000 Da, or 3,900 Da to 6,600 Da.

The first bonding portion may be connected to the magnetic screen by chemical bonding with a carboxylate group (—COO⁻) of the magnetic screen. The first bonding portion may include any one of an amino group (—NH₂) and a thiol group (—SH), but is not limited thereto and may include any one of functional groups capable of chemical bonding with the carboxylate group.

The second bonding portion may be connected to the substrate by chemical bonding with the thiol group (—SH) provided on the substrate. The second bonding portion may include any one or more of a maleimide group and an alkenyl group (—C═C—), but is not limited thereto and may include any one of functional groups capable of chemical bonding with the thiol group.

The linker may specifically have a structure of Formula 1 below. n in Formula 1 may be 30 to 5,000, preferably 90 to 5,000, or 90 to 150.

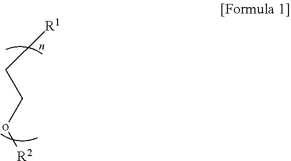

wherein R¹ is any one of an amino group (—NH₂) and a thiol group (—SH), and R² is any one of a maleimide group and an alkenyl group (—C═C—).

Preferably, Formula 1 may be Formula 2 below. n in Formula 2 may be 30 to 5,000, preferably 90 to 5,000, or 90 to 150.

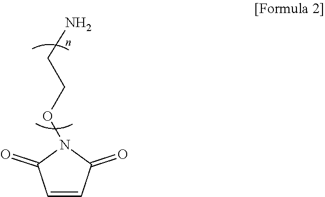

The maximum length of the linker may be such as a length that, when the linkers are elongated, entanglement between the linkers (or between the magnetic screens coupled to the linkers) may not occur or adhesion of stem cells to the ligands may not be interfered. In addition, the maximum length may be a sufficient length to block integrins from adhering to the ligands, when the linker is compressed. In addition, the minimum length of the linker may be such a length that, when the linker is elongated, a nanogap of sufficient size for integrins to adhere to the ligands may be formed, and when the linker is compressed, the ligands may be blocked by the magnetic screens so that stem cell integrins may not bind to the ligands. The length of the linker may be 10 nm to 1 pm, preferably 30 nm to 1 μm, or 30 nm to 50 nm.

One or more linkers may be bound to the magnetic screen.

At least one surface of the substrate may include a ligand.

The substrate may be formed by forming thiol groups on at least one surface of a glass substrate, chemically bonding a portion of the thiol groups to gold nanoparticles, and coupling ligands to the gold nanoparticles.

At least a portion of the thiol groups formed on the glass substrate may be chemically bonded to the gold nanoparticles, and ligands may not be bound to the thiol groups that have not been chemically bonded to the gold nanoparticles. The ligands may be RGD ligands.

At least a portion of the thiol groups on the glass substrate, which have not been chemically bonded to the gold nanoparticles, may be chemically bonded to the second bonding portions of the linkers.

The gold nanoparticles may be bound to the thiol groups while having a uniform distribution on the substrate. The gold nanoparticles may be added to cover 0.001% to 10% of the area of the substrate.

The magnetic screens, the linkers and the substrate may be connected to one another via chemical bonds to form the nanoscreen. The nanoscreen may be formed in a linear structure.

A surface of the magnetic screen, which faces the substrate, may be spaced apart from the ligand present on the substrate by a distance of a nanogap.

The nanogap may be reversibly changed by application of a magnetic field. The magnetic screen may move depending on the direction of application of the magnetic field, and the nanogap may be changed depending on the moving direction of the magnetic screen. When the magnetic screen is pulled in a direction away from the substrate by application of the magnetic field, the nanogap size may be increased as the linker is elongated. At this time, a space in which integrin can bind to the ligand on the substrate is formed (the ligand is unblocked), and thus integrin may bind to the ligand, thereby facilitating stem cell adhesion and differentiation. Conversely, when the magnetic screen is pulled in a direction toward the substrate by application of the magnetic field, the nanogap size may be reduced as the linker is compressed, the space in which integrin can bind to the ligand on the substrate disappears (the ligand is blocked), and thus integrin may not bind to the ligand, thereby inhibiting stem cell adhesion and differentiation.

Preferably, when the nanoscreen comprises the magnetic screens having the first average diameter, the stem cell adhesion and differentiation may be facilitated by elongating the linker and increasing the nanogap, through pulling of the magnetic screens in a direction away from the substrate by application of the magnetic field.

In addition, preferably, when the nanoscreen comprises the magnetic screens having the third average diameter, the stem cell adhesion and differentiation may be inhibited by compressing the linker and reducing the nanogap, through pulling of the magnetic screens in a direction toward the substrate by application of the magnetic field.

The magnetic screens may cover 68 to 80% of the area of the substrate. The density of the magnetic screens on the substrate may vary depending on to the size of the magnetic screens, but the percentage of the area of the substrate, which is covered by the magnetic screens, may be maintained constant. The maximum value at which the magnetic screens cover the area of the substrate may be in a range in which it is possible to form a space in which stem cells can adhere to the ligands, without interference between the magnetic screens in the process of regulating stem cell adhesion and differentiation using the nanoscreen. In addition, the minimum value at which the magnetic screens cover the area of the substrate may be in a range in which a sufficient amount of the magnetic screens may exist so that the magnetic screens can regulate stem cell adhesion and differentiation in the process of regulating stem cell adhesion and differentiation using the nanoscreen.

According to another embodiment of the present invention, the nanoscreen may be prepared by: forming aggregates of one or more magnetic particle units; forming a carboxylate group on the surfaces of the aggregates to form magnetic screens; binding each of the magnetic screens to one end of each linker by stirring the magnetic screens and the linkers; chemically binding the other end of each linker to thiol groups on a substrate on which thiol groups and ligands are present; and deactivating thiol groups on the substrate, which remain unbound to the linkers.

The magnetic particle units may be magnetic particles, and the magnetic screens may exhibit magnetism by including the magnetic particle units. The magnetic particle units may be combined together by hydrophobic interaction to form an aggregate. Specifically, an oil-in-water microemulsion may be prepared by suspending the magnetic particle units in chloroform and adding the suspension to a solution containing dodecyltrimethylammonium bromide (DTAB), and chloroform may be evaporated from the oil-in-water microemulsion by stirring, whereby an aggregate may be formed by hydrophobic interaction between the magnetic particle units. In the process in which close-packed nanoassembly of the magnetic particle units occurs, the hydrophobic surface of the aggregate of the magnetic particle units may be surrounded by amphiphilic DTAB, and then the aggregate may be stabilized by hydrophilic interaction with water. DTAB may form a micelle structure on the surface of the aggregate of the magnetic particle units. Therefore, the size of the aggregate may be adjusted by adjusting the amount of DTAB. Specifically, when the amount of DTAB is reduced, an area that may be surrounded by DTAB may be reduced, so that a large aggregate may be formed, thereby forming a magnetic screen having a large size. Conversely, when the amount of DTAB is increased, an area that may be surrounded by DTAB may increase, so that a small aggregate may be formed, thereby forming a magnetic screen having a small size.

The size of the aggregate may vary depending on the amount of the magnetic particle units combined together. The aggregate has a slightly polyhedral shape so that the inner angle formed by each face is large and the aggregate has a sufficiently large number of faces so to be close to a spherical shape.

According to the size of the aggregate, the diameter of the magnetic screens may be any one of a first average diameter, a second average diameter, and a third average diameter. The first average diameter may be 150 nm to 250 nm, the second average diameter may be 450 nm to 580 nm, and the third average diameter may be 650 nm to 900 nm. Preferably, the first average diameter may be 170 nm to 230 nm, the second average diameter may be 470 nm to 530 nm, and the third average diameter may be 670 nm to 830 nm.

The magnetic screen may be formed by providing a carboxylate group onto the surface of the aggregate. The carboxylate group may be formed by adding ethylene glycol containing poly(acrylic acid) (PAA) to a solution containing the aggregate.

The linker moiety may include a polyethylene glycol (PEG) portion, a first bonding portion, and a second bonding portion.

One end of the linker, which is chemically bonded to the magnetic screen, may be the first bonding portion. The first bonding portion may include any one functional group selected from among an amino group (—NH₂) and a thiol group (—SH), and may be connected to the carboxylate group of the magnetic screen by chemical bonding. The chemical bonding between the first bonding portion and the carboxylate group of the magnetic screen may be performed by mixing and stirring a solution containing the linkers and a solution containing the magnetic screens.

In addition, the other end of the linker, which is chemically bonded to the substrate, may be the second bonding portion. Here, thiol groups and ligands may be present on at least one surface of the substrate. The second bonding portion may include any one of a maleimide group and an alkenyl group (—C═C—), and may be connected to the thiol group on the substrate by chemical bonding. The chemical bonding between the second bonding portion and the substrate may be performed through a thiol-ene reaction.

After the magnetic screens, the linkers and the substrate are combined together, there may be unreacted thiol groups on the substrate, and the unreacted thiol groups may be deactivated. The deactivated thiol groups no longer react with the linkers, gold nanoparticles and ligands, and stem cells may not adhere thereto. The unreacted thiol groups may be deactivated by a compound such as the following Formula 3.

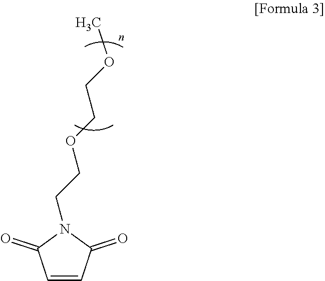

wherein n may be 10 to 5,000, preferably 10 to 30. The maleimide group in Formula 3 above may be chemically bonded to the unreacted thiol groups on the substrate through a thiol-ene reaction. This deactivation of the substrate makes it possible to block/unblock the ligands from stem cell adhesion using the magnetic screens.

The substrate may comprise a glass substrate, and thiol groups and ligands provided on at least one surface of the glass substrate. Here, the thiol groups may be provided by thiolating the glass substrate with mercaptopropylsilatran, and at least a portion of the thiol groups may be bound to gold nanoparticles, and the ligands may be bound to the gold nanoparticles bound to the thiol groups. A compound which is used to provide the thiol groups is not limited to mercaptopropylsilatran, and any compound capable of providing thiol groups on the glass substrate may be used without limitation.

The thiol groups may be formed by thiolating the glass substrate with mercaptopropylsilatran after activating the glass substrate with sulfuric acid. At least a portion of the thiol groups may be bonded to gold nanoparticles by Au—S bonding by incubation in a solution containing the gold nanoparticles. The gold nanoparticles may be bound to at least a portion of the thiol groups, but not all of the thiol groups. Thereafter, the substrate having the gold nanoparticles bound thereto may be incubated with a solution containing the ligand, so that the ligands may be bound to the gold nanoparticles. The ligands may be RGD ligands.

Another embodiment of the present invention includes a method of regulating stem cell adhesion and differentiation using a nanoscreen. Here, the nanoscreen may be the nanoscreen according to the embodiment described above.

The method of regulating stem cell adhesion and differentiation may comprise regulating stem cell adhesion and differentiation by applying a magnetic field to the nanoscreen. Specifically, the magnetic screens in the nanoscreen are magnetic and thus may be attracted in the direction in which the magnetic field is applied, and the nanogap may change depending on the direction in which the magnetic field is applied, so that stem cell adhesion and differentiation may be regulated. Here, the nanogap may be a distance between the surface of the magnetic screen, which faces the substrate, and the ligand present on the substrate. In other words, the nanogap may be a gap between the lower surface of the magnetic screen and the ligand when the substrate is assumed to be the bottom.

The magnetic field may be applied from outside the body to remotely control the nanoscreen in the body, thereby regulating stem cell adhesion and differentiation.

The magnetic field may be applied at a strength of 100 mT to 500 mT.

Specifically, the magnetic field may facilitate stem cell adhesion and differentiation by elongating the linker through pulling of the magnetic screens in a direction away from the substrate. When the linker is elongated, the nanogap, which is the gap between the magnetic screen and the ligand, may be increased (the ligand is unblocked), a space in which stem cells may adhere to the ligands may be formed, so that integrins of the stem cells may bind to the ligands, thereby facilitating stem cell differentiation.

In addition, specifically, the magnetic field may inhibit stem cell adhesion and differentiation by compressing the linker through pulling of the magnetic screens in a direction toward the substrate. When the linker is compressed, the nanogap may be reduced (the ligand is blocked), and the space in which stem cells may adhere to the ligands may disappear, so that binding of stem cell integrins to the ligands may be blocked, thereby inhibiting stem cell differentiation.

Hereinafter, examples of the present invention, comparative examples, and experimental examples will be described. However, the following examples are only preferred examples of the present invention, and the scope of the present invention is not limited by the following examples.

### EXAMPLES—PREPARATION OF NANOSCREEN

### Example 1

1. Preparation of Magnetic Screens

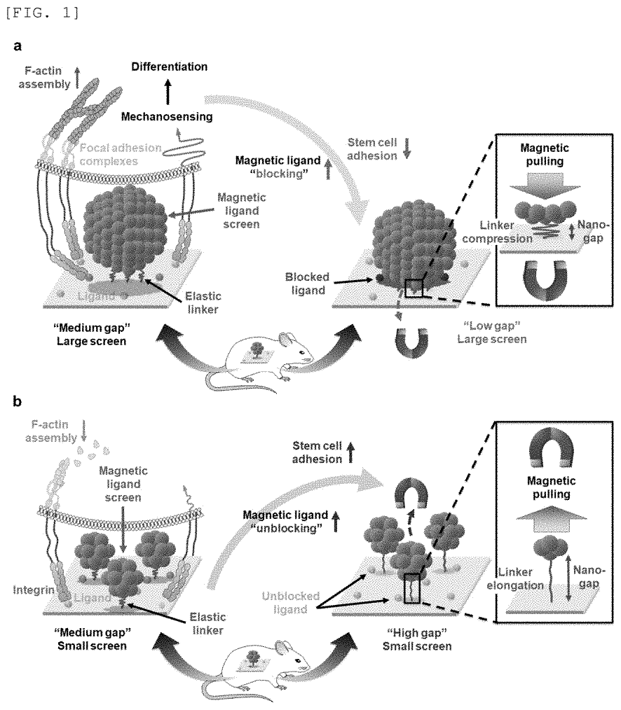

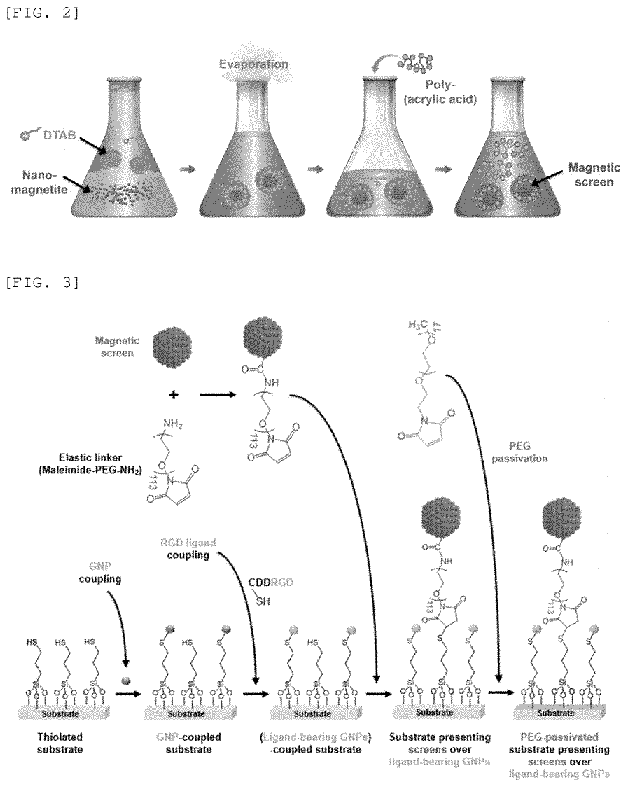

FIG. 2 schematically shows a process of preparing magnetic screens according to one embodiment of the present invention at a test tube scale.

Nano-magnetites were used as magnetic particle units for magnetic screens, and an aggregate was formed by performing closed-packed nanoassembly of the nano-magnetites.

First, 150 mg of nano-magnetites was suspended in 4.5 g of chloroform. This suspension was added to a solution containing 150 mg of dodecyltrimethylammonium bromide (DTAB) in 10 g of DI water, thus preparing an oil-in-water microemulsion. The oil-in-water microemulsion was agitated at room temperature for 16 h to direct the evaporation of chloroform, which assembles the nano-magnetites into 3-D structures via hydrophobic interaction of DTAB, thereby forming aggregates.

Next, the solution containing aggregates positively charged by DTAB present on their surface was added to 11.1 g of ethylene glycol containing 0.9 g of poly (acrylic acid) (PAA) to mediate coupling of the negatively charged PAA to the DTAB of the aggregates via electrostatic interactions. A carboxylate group was formed on the surfaces of the aggregates by coupling the PAA. Thereafter, the solution containing the aggregates having the carboxylate group was washed with deionized water, thus preparing 200-nm-sized magnetic screens.

2. Preparation of Substrate

(1) Preparation of Gold Nanoparticles

1 mM gold (III) chloride trihydrate was added to 50 mL of DI water and vigorously stirred at 100° C. for 20 minutes, thus preparing gold nanoparticles (GNPs). Subsequently, 38.8 mM of sodium citrate tribasic dihydrate in 5 mL of DI water was added to this solution, which was then subjected to vigorous stirring for 10 min. When the solution exhibited a red color, it was cooled down to room temperature and washed with sodium citrate-containing DI water to obtain a gold nanoparticle ((GNP) suspension. The prepared gold nanoparticles prepared can be identified by HR-TEM in c of FIG. 4. Here, the scale bar is 2 nm.

(2) Thiolation of Substrate

Prior to coupling ligands to a substrate, the substrate was thiolated.

First, glass substrates (22×22 mm) were immersed in a mixture of HCl and methanol (1:1) for 30 min to remove contaminants on the surface, followed by rinsing with DI water. Subsequently, the substrates were incubated in sulfuric acid for 1 h to activate the substrate surface for thiolation, followed by serial rinsing with DI water and methanol. Then, the substrates were thiolated with 0.5 mM mercaptopropylsilatrane in methanol for 1 h in dark conditions, followed by serial rinsing with methanol and DI water.

(3) Ligand Coupling to Thiolated Substrate

A diluted GNP solution was prepared by mixing the GNP suspension prepared in (1) with DI water containing 20 nM sodium citrate at a ratio of 1:200. The thiolated substrates prepared in (2) above were placed in 1.7 nM of the GNP solution and incubated at room temperature for 2 hours to graft the gold nanoparticles onto the thiolated substrates via Au—S bonding. The gold nanoparticle-grafted substrates were then serially rinsed with sodium citrate-containing DI water followed by pure DI water. The gold nanoparticle-grafted substrates were further incubated in DMSO (dimethyl sulfoxide) containing 0.2 nM thiolated RGD peptide (CDDRGD), 0.2% of N,N-diisopropylethylamine (DIPEA), and 10 mM tris(2-carboxyethyl)phosphine hydrochloride (TCEP) at room temperature for 12 h in dark conditions, followed by rinsing with DI water.

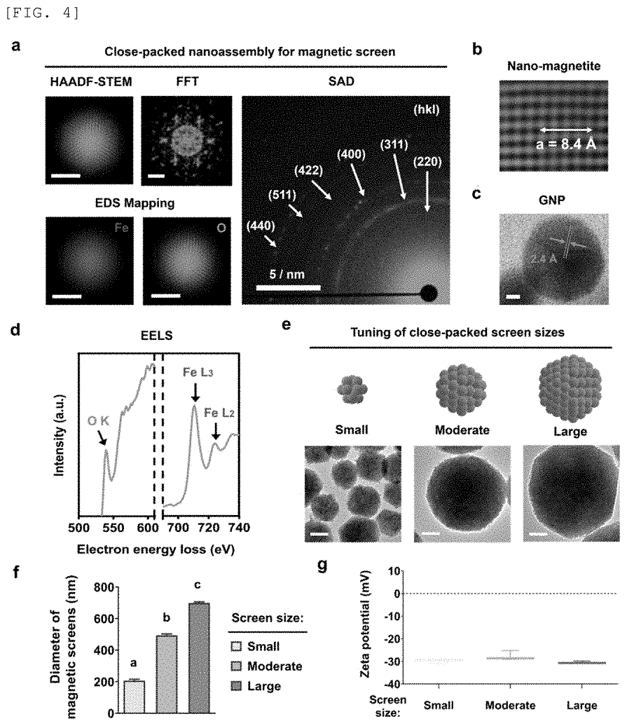

(4) Tethering of magnetic screens to substrate Elastic maleimide-poly(ethylene glycol)-NH₂ (Mal-PEG 5 kDa-NH₂) was used as linkers to tether the magnetic screens prepared in 1 above to the substrate prepared in 2. The length of the linker used is about 38.2 nm. FIG. 3 schematically shows a process of coupling the magnetic screen to the linker, followed by coupling to the ligand-coupled substrate.

The carboxylate group on the magnetic screens was allowed to react with the amine group of Mal-PEG-NH₂ via the EDC/NHS reaction. The magnetic screens were added at a concentration of about 8.6×10⁹ per mL of solution. First, 0.5 mL of DI water containing the magnetic screens was allowed to react with 0.5 mL of DI water containing 1.4 mg of N-ethyl-N′-(3-(dimethylaminopropyl))carbodiimide) (EDC), 6.4 mg of N-hydroxy-succinimide (NHS), 0.4 mg of Mal-PEG-NH₂ and 0.2% (v/v) of N,N-di-isopropyl-ethylamine (DIPEA). The suspension was vigorously stirred for 16 h under dark conditions to form PEGylated magnetic screens. Thereafter, 1 mL of the PEGylated magnetic screens (linker-coupled magnetic screens) were elastically tethered to the substrates decorated with RGD ligand-coated GNPs via the thiol-ene reaction for 16 h under dark conditions. Through the elastic tethering of the magnetic screens to the substrate, nanogaps were formed between the magnetic screens and the underlying ligands. After washing the substrate with DI water, the thiolated surface of the substrate that had not reacted with the GNPs or PEGylated magnetic screens was passivated by treatment with 1 mL of DI water containing 100 μM of methoxy-PEG (2 kDa)-maleimide for 2 h under dark conditions via the thiol-ene reaction, followed by rinsing with DI water.

As a result, a nanoscreen including about 200-nm-sized magnetic screens was prepared.

### Examples 2 and 3

Nanoscreens were prepared in the same manner as in Example 1 under conditions different from those of Example 1.

In order to maintain the density of ligands that are not covered by the magnetic screens in the nanoscreen, when the size of the magnetic screens increased during the EDS/NHS reaction, the concentration of the reacting magnetic screens was reduced to reduce the density of the magnetic screens tethered to the substrate.

In Example 2, during the process of preparing the magnetic screens, an oil-in-water microemulsion was prepared using 10 g of DI water containing 100 mg of DTAB. In the process of tethering the magnetic screens to the substrate, the magnetic screens were added at a concentration of about 1.1×10⁹ per mL of solution. As a result, a nanoscreen including about 500-nm-sized magnetic screens was prepared.

In Example 3, during the process of preparing the magnetic screen, an oil-in-water microemulsion was prepared using 10 g of DI water containing 50 mg of DTAB. In the process of tethering the magnetic screen to the substrate, the magnetic screens were added at a concentration of about 0.4×10⁹ per mL of solution. As a result, a nanoscreen including 700-nm-sized magnetic screens was prepared.

### Comparative Examples

For comparison with the effects of the Examples, nanoscreens of Comparative Examples were prepared.

The nanoscreen of Comparative Example 1 was prepared in the same manner as Example 1, except that the ligands were not coupled to the substrate.

The nanoscreen of Comparative Example 2 was prepared in the same manner as Example 1, except that the magnetic screens and the linkers were not used.

### Experimental Examples

In the experimental examples and the drawings, the 200-nm-sized magnetic screens or aggregates may be denoted as small, and the nanoscreen of Example 1 may be denoted as small (or small nanoscreen). In addition, in the experimental examples and the drawings, the 500-nm-sized magnetic screens or aggregates may be denoted as moderate, and the nanoscreen of Example 2 may be denoted s moderate (or moderate nanoscreen). In addition, in the experimental examples and the drawings, the 700-nm-sized magnetic screens or aggregates may be denoted as large, and the nanoscreen of Example 3 may be denoted as large (or large nanoscreen).

In addition, a nanogap in a state in which no magnetic field is applied to the nanoscreen is denoted as a medium gap, a nanogap in a state in which a magnetic field is applied so that the magnetic screen is pulled in a direction away from the substrate is denoted as a “high gap”, and a nanogap in a state in which a magnetic field is applied so that the magnetic screen is pulled in a direction toward the substrate is denoted as a “low gap”. In addition, a state in which a “high gap” state is formed by a magnetic field so that a stem cell (or integrin) may adhere to a ligand is expressed as “the ligand is unblocked”, and a state in which a “low gap” state is formed so that a stem cell (or integrin) may not adhere to a ligand is expressed as “the ligand is unblocked”.

### Experimental Example 1—Analysis of Magnetic Screens

a to g of FIG. 4 show the results of analyzing the characteristics of the magnetic screens prepared in Example. An experimental method for each figure is as follows.

1. HAADF-STEM (High-Angle Annular Dark-Field Scanning Transmission Electron Microscopy of Aggregate

a of FIG. 4 includes a HAADF-STEM image of a magnetic screen. To analyze the close-packed “nanoassembly” of nano-magnetites (Fe₃O₄), which are magnetic particle units in an aggregate constituting a magnetic screen, at the nanometer level and analyze the close-packed “atomic arrangement” within the nano-magnetite at the atomic level, HAADF-STEM was conducted. Measurements were carried out at 200 kV using a probe Cs-corrected JEM ARM200CF (JEOL Ltd.) under a spherical aberration (C3) of 0.5 to 1 μm and a measured phase of 27 to 28 mrad. The convergence semi-angle for imaging and the collection semi-angle for HAADF were 21 and 90 to 370 mrad, respectively. Imaging was carried out with electron probe sizes of 8 C (1.28 Å) and 9 C (1.2 Å) (JEOL defined). In addition, a pixel dwelling time of 10 to 15 μs, a pixel area of 2048×2048, an emission current of 8 to 13 μA, and a probe current range of 10 to 20 μA were also used. HAADF-STEM imaging confirmed the periodic arrays of close-packed nanoassembled nano-magnetite units. From the HAADF-STEM image, the unit cell lattice parameter of the nano-magnetite was calculated to be 8.4 Å (b of FIG. 4). In the HAADF-STEM image of a of FIG. 4, the scale bar is 100 nm.

2. Energy-Dispersive X-Ray Spectroscopy (EDS) Mapping of Aggregate

a of FIG. 4 also includes EDS mapping. To identify the dual presence of Fe and O elements in the magnetic screens, EDS mapping was carried out using two SDD detectors (Thermo Fisher Scientific). It was confirmed that Fe and O elements homogeneously distributed in the magnetic screens were selectively identified to confirm the dual presence of Fe and O elements in the aggregate. In the EDS mapping image of a of FIG. 4, the scale bar is 100 nm.

3. Fast Fourier Transform (FFT) to Confirm Crystallographic Structure of Magnetic Particle Unit

a of FIG. 4 also includes an FFT image. To confirm the crystallographic nanostructures of the aggregate, FFT was utilized. In this experiment, the FFT was applied to the HAADF-STEM images at intermediate magnification such that the pattern array contains information on the overall configuration of the aggregate. Since the HAADF-STEM images were taken in the precisely aligned direction along the molecular zone axis (not the atomic zone axis), the FFT generated from the HAADF-STEM confirmed the nature of the close-packed nanoassembly. The hexagonal array spots in the FFT correspond to the close-packed assembly of the magnetic particle units because hexagonal arrays can only originate from close-packed molecular structures. Here, the scale bar is 0.1/nm.

4. Selected Area Diffraction (SAD) to Confirm Crystallographic Structure of Magnetic Particle Unit

a of FIG. 4 also includes a SAD pattern. To verify the atomic crystal structure of the nano-magnetite particles which are magnetic particle units in the 200-nm-sized magnetic screen, SAD pattern was exploited. Various SAD patterns were obtained at different camera lengths, with those at a camera length of 6 cm being utilized to collect crystallographic information on the nano-magnetite particles. The SAD pattern showed a series of concentric diffraction rings identified as the (220), (311), (400), (422), (511), and (440) diffraction planes, which indicates the random orientation of the nano-magnetites (Fe₃O₄). The interplanar d-spacings of the (220), (311), (400), (422), (511), and (440) diffraction planes were measured as 2.97, 2.53, 2.09, 1.72, 1.61, and 1.48 Å, respectively. These values were compared with previously reported values of 2.969, 2.532, 2.099, 1.714, 1.616 and 1.484 Å, respectively. Other peaks, such as (111) and (222) diffraction planes were present but not distinct, thereby implying that these planes exhibit slight atomic disorderings. Here, the scale bar is 5/nm.

5. Electron Energy Loss Spectroscopy (EELS) to Confirm the Presence of Fe and O in Aggregate

d of FIG. 4 shows the results of EELS of the magnetic screens.

To cross-check the dual presence of Fe and O elements in the magnetic screens, EELS was performed. In this experiment, the magnetic screens having a size of 200 nm were used. Measurements were carried out at 200 kV using a probe Cs-corrected JEM ARM200CF (JEOL Ltd.) equipped with a Gatan K2 summit direct electron detector. In addition, measurements were performed in both electron counting and 965 GIF Quantum ER in Dual EELS modes to establish the correct edge energy calibration from the zero loss peak (ZLP). The EELS spectra showed peaks for Fe L₃, L₂ (715 and 730 eV), and O K (540 eV) in the magnetic screens, which confirmed the dual presence of Fe and O elements in the close-packed nanoassembly of nano-magnetites (Fe₃O₄).

6. Transmission Electron Microscopy (TEM) to Form Aggregate Size

e of FIG. 4 Shows TEM Images of the Aggregates, and f of FIG. 4 is a graph showing the results of quantifying the sizes of the aggregates. To visualize the sizes, shapes, and monodispersities of magnetic screens of various sizes, TEM imaging was carried out using Tecnai 20 (FEI, USA). Using ImageJ software from four different images, the sizes of the aggregates were calculated. As shown in e of FIG. 4, the aggregates have a slightly polyhedral shape, but can be considered almost spherical. The sizes of the aggregates were measured as 206.5±16.2 nm (small), 493.2±15.5 nm (moderate) and 698.1±11.8 nm (large). These sizes correspond to 200 nm, 500 nm and 700 nm to be prepared in the Example. Here, the scale bar is 100 nm.

In addition, dynamic light scattering (DLS) was performed for size distribution profiles (hydrodynamic diameters) of magnetic screens having various sizes. In this analysis, the average hydrodynamic diameters of the magnetic screens were measured to be 210.7±20.9 nm (small), 513.7±39.2 nm (moderate), and 760.2±89.2 nm (large), respectively. These also correspond to the hydrodynamic diameters to be prepared in the Example.

7. Confirmation of Carboxylate Functional Group in Magnetic Screens

g of FIG. 4 shows the results of performing zeta potential analysis of magnetic screens having various sizes using Zetasizer Nano ZS90 Malvern Panalytical (Malvern, UK). The magnetic screens are negatively charged by the carboxylate group formed in the magnetic screens, and the zeta potential values were measured as −29.8±0.9 mV (small), −27.6±2.1 mV (moderate) and −30.5±0.6 mV (large), respectively. These numerical results indicate that the carboxylate group was successfully bound to the magnetic screens. In the graph, n=3, and data are presented as mean±standard error.

8. Analysis of Magnetic Characteristics of Magnetic Screens

FIG. 5 shows hysteresis loops for confirming the magnetic properties of magnetic screens having various sizes. In this experiment, vibrating sample magnetometry (VSM) (EV9; Microsense) was performed at 300 K. The hysteresis loop of the magnetization (M) in response to the application of a magnetic field (H) was obtained after normalization to the maximum value for each magnetic screens.

### Experimental Example 2—Analysis of Nanoscreen Morphology

After the nanoscreens were prepared, the morphologies of the nanoscreens were analyzed.

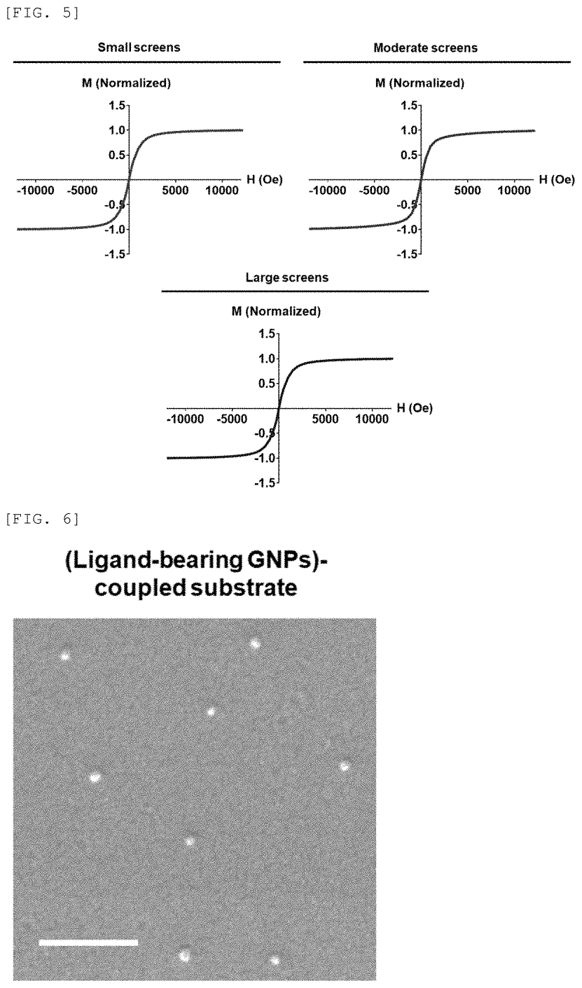

FIGS. 6, 7 and 8 show that the magnetic screens in the nanoscreens are uniformly distributed with respect to the ligand in the nanoscreen.

Field emission scanning electron microscopy (FE-SEM) imaging (FEI, Quanta 250 FEG) was performed on nanoscreens having magnetic screens of different sizes. A substrate was subjected to drying and platinum coating using a sputter coater prior the FE-SEM imaging. Using ImageJ software for four different images, the substrate-coupled ligand density was calculated in the absence of the elastic tethering of the magnetic screens and presented as particles/pmt. The substrate-tethered screen density (increasing with decreasing screen sizes) was calculated and presented as particles/μm². In addition, the total area blocked by the substrate-tethered screens (similar for the various screen size groups) was calculated per total surface area and presented as the relative percentage (%). Magnetic screens were prepared at concentrations of 8.6×10⁹ (small), 1.1×10⁹ (moderate) and 0.4×10⁹ (large) magnetic screens per mL, respectively, and tethered to substrates at densities of 6.4±0.6 (small), 1.8±0.3 (moderate) and 0.8±0.1 (large) magnetic screens/μm², respectively.

FIG. 6 is an image in which ligand-bearing GNPs are coupled to a substrate. It can be seen that the ligands are spaced apart at almost uniform intervals. Here, the scale bar is 200 nm.

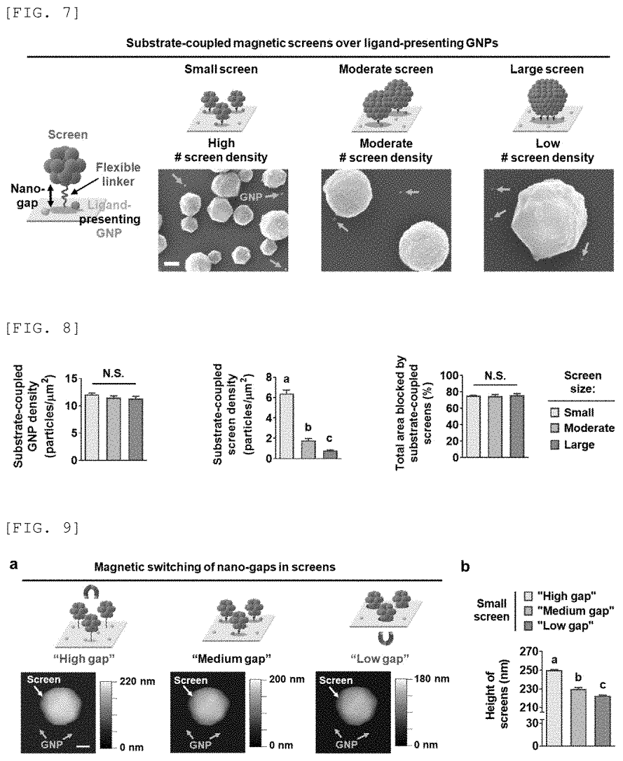

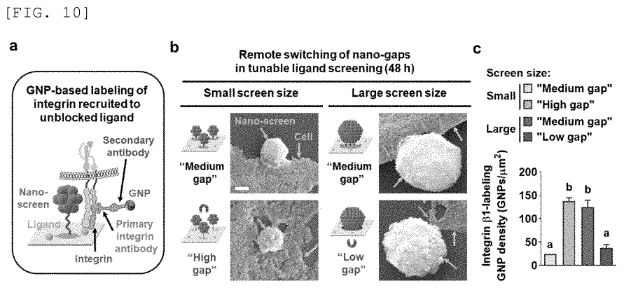

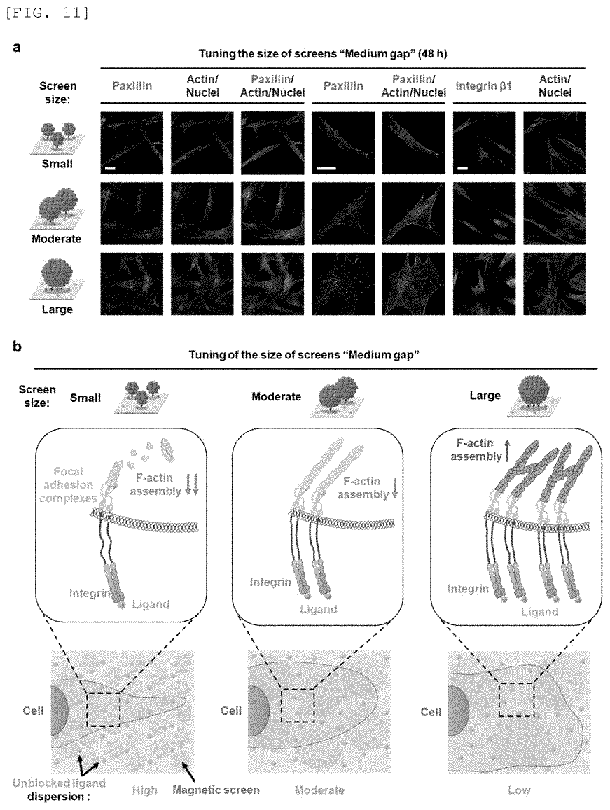

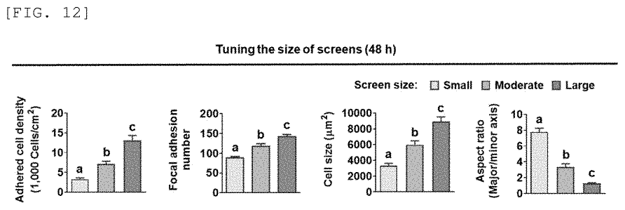

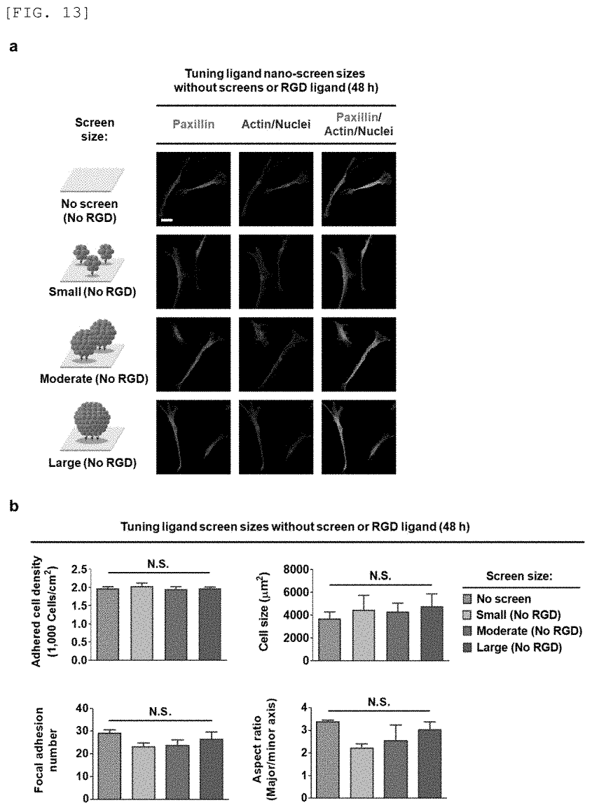

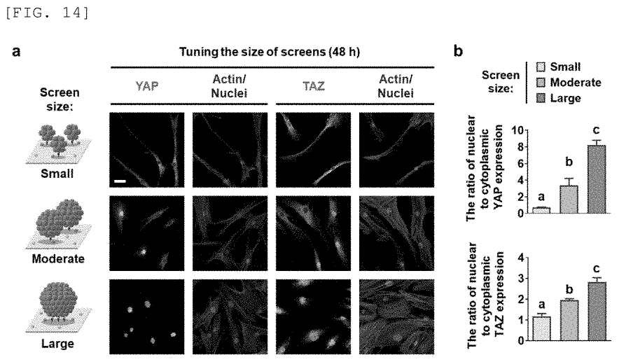

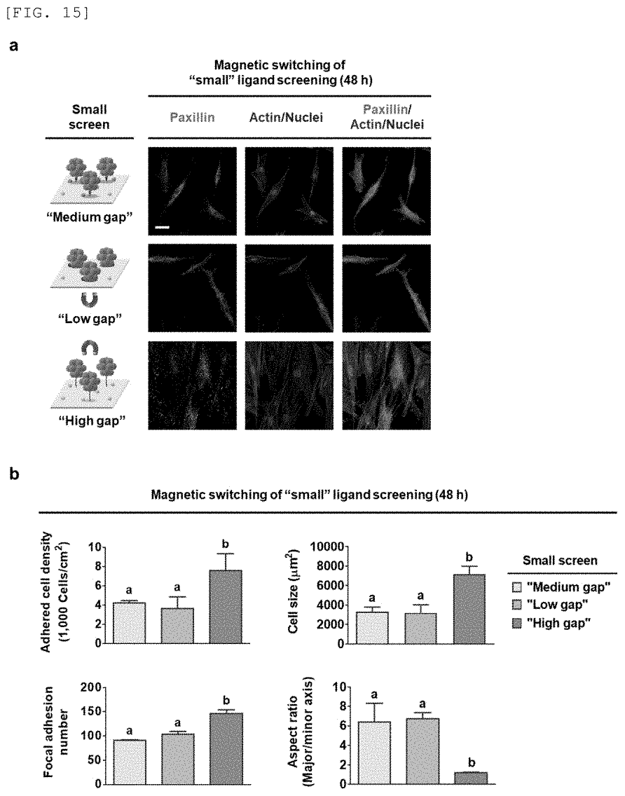

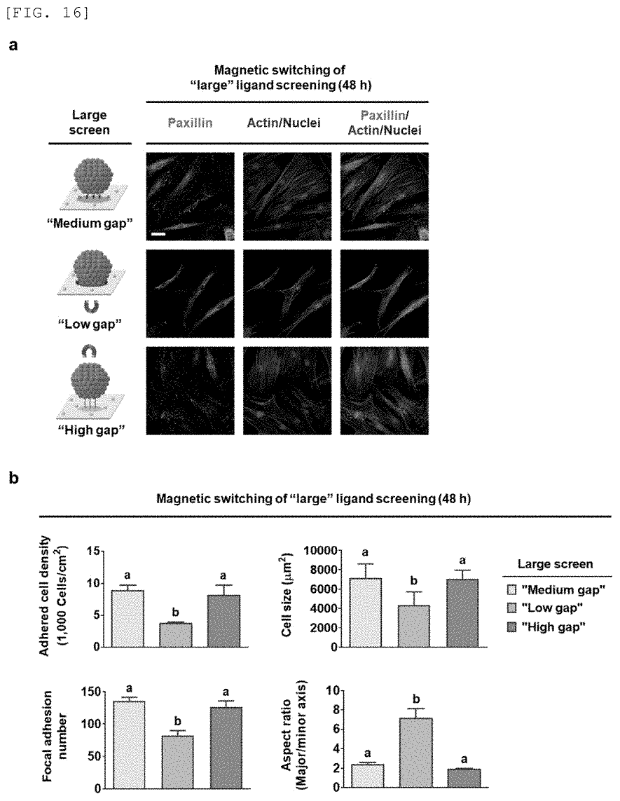

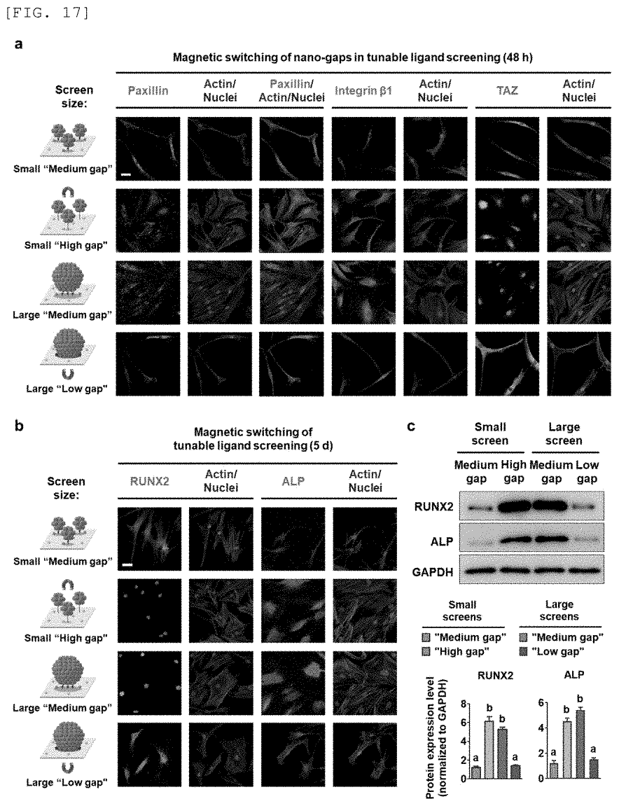

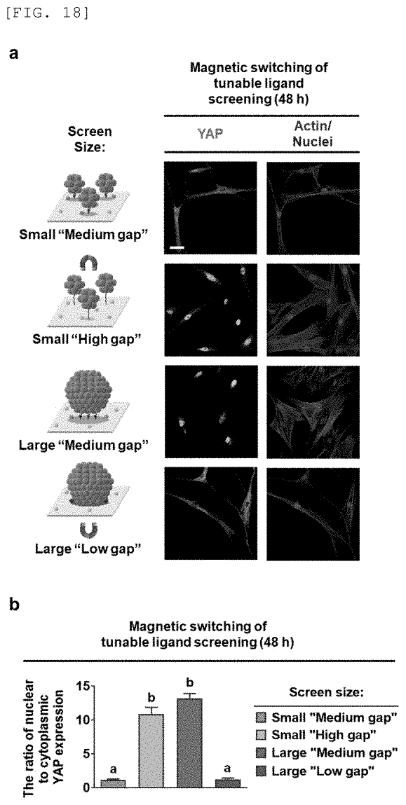

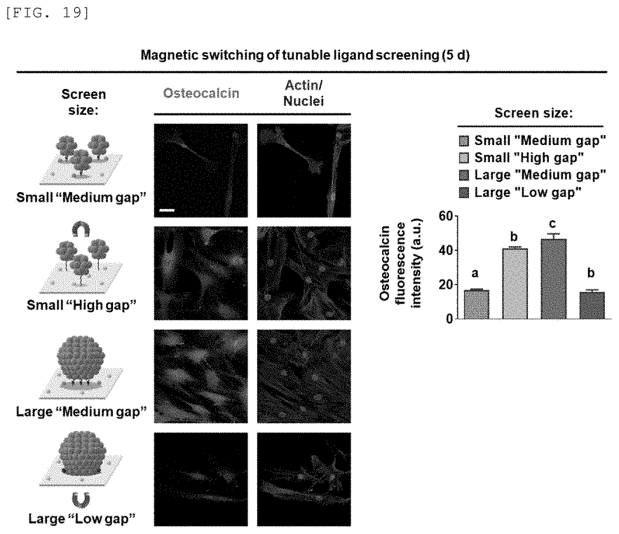

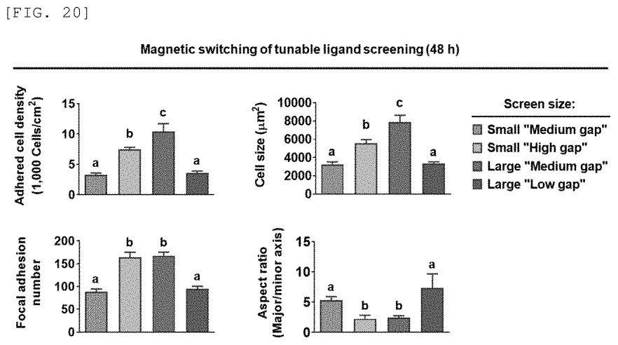

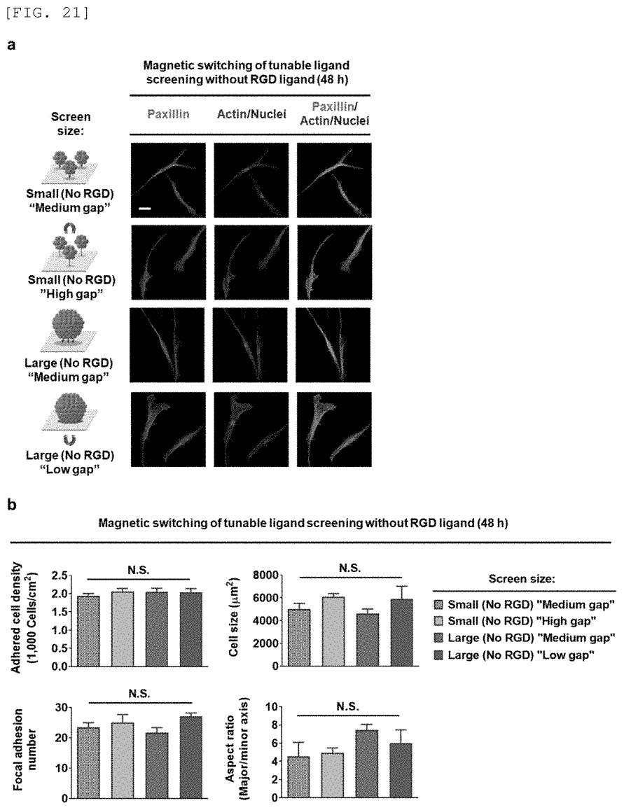

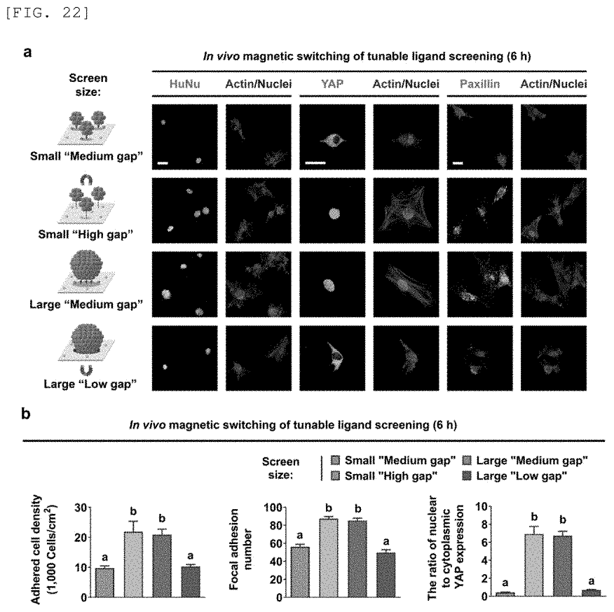

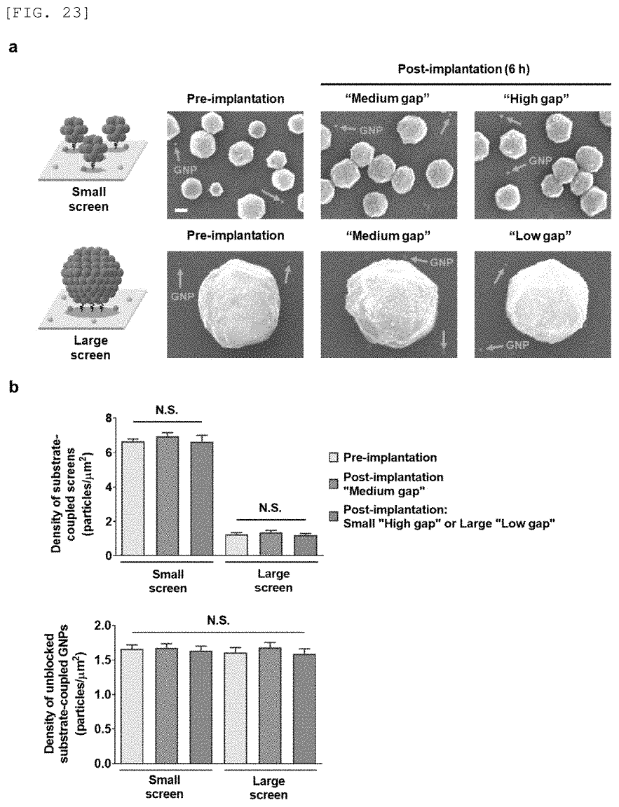

In FIG. 7, yellow arrows indicate ligand-bearing GNPs. In the images, it can be seen that the ligand-bearing GNPs are present in an almost uniform ratio regardless of the sizes of the magnetic screens on the substrates. In addition, in FIG. 8, it can be seen that the substrate areas covered by tethering of the magnetic screens are calculated as 75.2% (small), 75.0% (moderate), and 74.2% (large), which are almost uniform.

Taking the results in FIGS. 6 and 8 together, it is obvious that the proportion of the ligands covered by the magnetic screens in FIG. 7 is almost uniform. As about 75% of the area of the substrate is covered by the magnetic screens, it is possible to finely regulate cell adhesion by adjusting the nanogap.

### Experimental Example 3—Analysis of Change in Nanogap Size by Application of Magnetic Field

An experiment was performed to confirm that the nanogap size was changed by applying a magnetic field to the nanoscreen. a of FIG. 9 depicts schematic views and atomic force microscopy (AFM) images showing the changes in the nanogap size by the application of a magnetic field to the small nanoscreen. b of FIG. 9 is a graph showing the results of quantifying the sizes of these nanogaps. Since the size changes of the nanogaps depend on the length of the linker and show a similar value regardless of the size of the magnetic screens, only the small nanoscreen is shown in the figure. Here, the nanogap is a distance between the magnetic screen surface facing the substrate and the ligand.

a of FIG. 9 shows the results of performing magnetic AFM imaging (Asylum Research, XE-100 System). For example, magnetic pulling upward of the magnetic screens via elongation of the elastic linker increases the nanogaps between the magnetic screens and substrate, thereby facilitating the relative unblocking of the underlying ligand. Conversely, magnetic pulling downward of the magnetic screens via compression of the elastic linker decreases the nanogaps, thereby facilitating the relative blocking of the underlying ligand. An AFM cantilever (Nanosensors, SSS-SEIHR-20, spring constant: 5 to 37 N/m and resonance frequency: 96 to 175 kHz) was used for the imaging in AC conditions using the air mode at room temperature. Serial magnetic AFM imaging in situ was performed on the same scanned area on the substrate presenting magnetic screens over ligand-bearing GNPs while the magnetic screens were pulled upward or downward by placing a permanent magnet (300 mT) at the upper (“high gap”) or lower (“low gap”) side of the substrate, respectively, or else remained stationary (“medium gap”) without a the magnet. In a of FIG. 9, yellow arrows (GNPs) are ligand-bearing gold-nanoparticles, which are not covered by the magnetic screens.

As a result of the measurement, the heights of the magnetic screens were 229.7±3.1 nm in the high gap, 250.0±1.0 nm in the medium gap, and 222.7±1.2 nm in the low gap (b of FIG. 9). These values are the results obtained by measuring the distance from the upper surface of the magnetic screen to the substrate when the substrate unit was placed at the bottom. Therefore, it can be confirmed that the nanogap can be adjusted by applying a magnetic field to the nanoscreen. In addition, the nanogap can be indirectly calculated based on the measurement results.

### Experimental Example 4—Examination of Cell Adhesion Depending on Nanogaps

In order to check whether there is a difference in the degree of stem cell adhesion depending on the size of the nanogap, single-cell level imaging and quantification of integrin recruited to the ligand were performed through immunogold labeling. The results are shown in a to c of FIG. 10. The present inventors conducted experiments a small nanoscreen and a large nanoscreen and used secondary antibody-coated GNPs (40 nm) to immunogold-label primary antibody (integrin β1)-coated human mesenchymal stem cells. Primary antibody (integrin β1)-coated human mesenchymal stem cells were added only at 0 h at the start of the experiment. A schematic view for this experiment is shown in a of FIG. 10.

b of FIG. 10 depicts FE-SEM images showing the state 48 hours after the start of the experiment, and c of FIG. 10 shows the results of quantifying the density of the recruited integrin. In the images, nanoscreens are indicated by blue arrows, immunogold-labeled integrin is indicated in yellow, and stem cells are indicated in magenta. Although the ligand density was the same in the figure, it could be confirmed that integrin recruitment was more facilitated in the large nanoscreen than in the small nanoscreen under the medium nanogap condition. Furthermore, ut could be confirmed that the small nanoscreen exhibited a greater effect (than nanoscreens of other sizes) on integrin recruitment to the ligand by the formation of “high gap”, and the large nanoscreen exhibited a greater effect (than nanoscreens of other sizes) on blocking of the ligand by the formation of “low gap”. In the images, the scale bar is 100 nm.

### Experimental Example 5—Regulation of Stem Cell Adhesion and Differentiation by Magnetic Screens

An experiment was conducted as to whether stem cell adhesion and differentiation could be regulated depending on the presence of the magnetic screens and the ligand and the size of the magnetic screens.

Ultraviolet light irradiation was applied to the substrate for 2 h to sterilize it, and then human mesenchymal stem cells (hMSCs at the passage 5 from Lonza) were plated at a density of 10,500 cells per cm² in growth medium with high glucose Dulbecco's Modified Eagle Medium (DMEM) supplemented with 10% fetal bovine serum (FBS), 4 mM of L-glutamine, and 50 U/mL of penicillin and streptomycin at 37° C. under 5% CO2. To confirm focal adhesion and mechanotransduction, stem cells were cultured for 48 hours in medium gap conditions to which magnetic screens of various sizes were applied. For these cultured cells, focal adhesion and mechanotransduction of the cultured stem cells was analyzed while pulling the magnetic screens upward or downward by placing the magnet (300 mT) at the upper (“high gap”) or lower (“low gap”) side of the substrate throughout the entire culture time of 48 h. In addition, substrates with no RGD ligand with and without magnetic screens were used to confirm the requirement of the RGD ligand to be blocked by the magnetic screens of various sizes to effectively regulate stem cell adhesion. The focal adhesion-aided mechanotransduction of stem cells was explored for small screens (“medium gap” and “high gap”) and large screens (“medium gap”) after 48 h of culturing in growth medium supplemented with 2 μg/mL cytochalasin D for actin polymerization inhibition, 50 μM Y27632 for ROCK inhibition, or 10 μM blebbistatin for myosin II inhibition. The focal adhesion-mediated differentiation of stem cells was explored for small screens (“medium gap” and “high gap”) and large screens (“medium gap” and “low gap”) after 5 days of culturing in osteogenic medium (growth medium supplemented with 100 nM dexamethasone, 10 mM β-glycerophosphate, and 50 μM ascorbic acid-2-phosphate).

Cellular integrin recruitment on the magnetic screens under magnetic switching of the nano-gaps of the magnetic screens was examined via immunogold labeling at the single cell level. The present inventors used GNPs of 40 nm in diameter (different from the 10 nm GNP used to coat RGD ligand on the material surface) to label integrin recruited to the unblocked ligand. GNPs (40 nm) were incubated in secondary antibody (goat anti-mouse (H+L) IgG (1:100), Abcam) in blocking buffer containing 1% bovine serum albumin (BSA) and 0.1% Tween 20 in 0.1 M 1,4 piperazine bis(2-ethanosulfonic acid) (PIPES) buffer (pH=7.4) under mild shaking at 37° C. for 16 h. At 48 h of culturing, stem cells on the nanoscreen were first rinsed with 0.1 M PIPES buffer (pH=7.4) for 2 min. The rinsed cells were then fixed with 4% paraformaldehyde for 15 minutes followed by rinsing with PBS. The fixed cells were permeabilized with 0.5% Triton X-100 in a buffer solution (pH=7.2) containing sucrose, NaCl, MgCl₂ and HEPES in DI water for 1 min. The permeabilized cells were treated with blocking buffer for 1 h. The blocked cells were incubated in primary antibody against integrin β1 (mouse) in the blocking buffer at 37° C. for 2 h followed by rinsing with 1% BSA and blocking with 5% goat serum for 15 min. The cells treated with primary antibody against integrin β1 were then incubated with secondary antibody-conjugated GNPs in PIPES buffer at 25° C. for 16 h. The cells were rinsed with PIPES buffer and completely fixed with 2.5% glutaraldehyde solution for 5 min, followed by rinsing with PIPES buffer. To elevate the imaging contrast, the cells were then treated with 1% osmium tetroxide in PIPES buffer for 1 h, followed by rinsing with PIPES buffer and DI water. After drying, the cellular integrin β1 labeled with the GNPs was imaged via FE-SEM. This FE-SEM image was used to visualize the integrin (yellow-pseudo-colored integrin-GNP) recruited to the unblocked ligand in a single cell (magenta-pseudo-colored). The number of integrin-GNPs per unit area was also quantified.

a of FIG. 11 shows that the adhesion of paxilin, F-actin, and integrin β1 of stem cells increases in the “medium gap” state 48 hours of culture as the size of the magnetic screens increases, and b of FIG. 11 schematically shows that cell adhesion occurs in the “medium gap” state depending on the size of the magnetic screens. Here, the scale bar is 50 μm. FIG. 12 shows the results of quantifying this cell adhesion, and it can be seen that, as the magnetic screen size increased, the cell aspect ratio decreased, but the adherent cell density, focal adhesion number, cell size, and cell aspect ratio increased. These findings prove that elevated integrin recruitment and expression stimulates the focal adhesion of stem cells more with increasing screen size.

a and b of FIG. 13 show the results of an experiment on whether the adhesion of stem cells occurs well when there is no ligand or magnetic screen in the nanoscreen. As compared with a and b of FIG. 11 and FIG. 12, in a of FIG. 13, it can be seen that paxillin, actin, and nuclei significantly decreased. Also, from the quantification graphs in b of FIG. 13, it can be seen that the numerical values significantly decreased in all aspects, and when there was no ligand, the numerical values were almost constant regardless of the size of the magnetic screens. Therefore, it can be confirmed that both the magnetic screens and the ligand are required for stem cell adhesion and expression. N.S. in b of FIG. 13 indicates that there is no particular difference between the comparison groups.

a and b of FIG. 14 depict images and quantification graphs showing the results of conducting experiments depending on the size of magnetic screens in a state in which no magnetic field is applied. It can be confirmed that the ratio of nuclear-to-cytoplasmic expression of YAP and TAZ, which are co-regulators of mechanotransduction, significantly increased as the size of the magnetic screens increased. These results prove that modulating the screen size alone without changing the unblocked ligand density can effectively regulate the focal adhesion and mechanotransduction of stem cells.

### Experimental Example 6—Test for Regulation of Stem Cell Adhesion and Differentiation by Change in Nanogap Size

It was tested whether the adhesion and differentiation of stem cells were changed by tuning the nanogaps through the application of a magnetic field. Focal adhesion-mediated mechanotransduction and differentiation of stem cells were analyzed via immunofluorescence staining while tuning the nanogaps of the nanoscreens. After magnetic switching of the nanogap size, stem cells were treated with fixing solution (4% paraformaldehyde) at 25° C. for 12 min and washed with phosphate-buffered saline (PBS). The cells were then subjected to permeabilization in blocking solution (PBS containing 3% bovine serum albumin with 0.1% Triton X-100) at 25° C. for 30 min. Primary antibodies were added to the blocking solution in which the stem cells were then incubated at 4° C. for 16 h, followed by washing with PBS containing 0.5% Tween 20. Fluorochrome-tagged secondary antibodies with phalloidin and DAPI were added to the blocking solution in which stem cells were immersed at 25° C. for 30 min, followed by washing with PBS containing 0.5% Tween 20. Confocal laser scanning microscopy (LSM700, Carl Zeiss) was used to obtain images of the immunofluorescently stained stem cells by applying identical exposure conditions to all of the compared groups, followed by analysis with ImageJ software. Focal adhesion, mechanotransduction, and differentiation of stem cells were quantified by using Image J software on the images of immunofluorescently stained stem cells. The adhered stem cell density per unit area was determined by counting the number of cell nuclei from four different DAPI-stained images. Focal adhesion was quantified by counting paxillin-positive areas exceeding 1 μm² in 10 stem cells from four different images. The ratios of nuclear to cytoplasmic fluorescent intensities of YAP, TAZ, and RUNX2 in stem cells were calculated to estimate their nuclear localization. Fluorescence intensities were also calculated for the expression of alkaline phosphatase (ALP) and osteocalcin.

In addition, Western blotting-based quantitative analysis of stem cell differentiation influenced by magnetic switching of the nanogaps was performed. Stem cell differentiation on substrates with tunable magnetic screens under magnetic control of the nanogaps was analyzed via Western blotting analysis. After being subjected to magnetic switching of the nanogaps, stem cells in osteogenic medium were examined after 5 days of culturing for small screens (“medium gap” and “high gap”) and large screens (“medium gap” and “low gap”). The cells were collected in PRO-PREP™ protein extraction solution buffer containing a protease inhibitor cocktail for 20 min and then centrifuged at 4° C. The total protein concentration of the supernatant was measured by using a BCA protein assay (Thermo Scientific™ Pierce™ BCA Protein Assay Kit). The protein samples were mixed with a loading dye and denatured by boiling at 100° C. for 10 min. The denatured proteins were then separated by electrophoresis using 10% SDS-PAGE-gels at 110 V for 1 h and transferred to a polyvinylidene fluoride (PVDF) membrane for further electrophoresis at 120 V for 90 min. The membranes were blocked with a blocking buffer (TBST containing 5% skim milk) at 4° C. for 16 h. The membranes were then rinsed with TBST and further incubated with anti-His-HRP secondary antibodies (diluted in blocking buffer) at room temperature for 1 h followed by thorough rinsing with TBST. Chemiluminescence signals were recorded using a Linear ImageQuant LAS 4000 mini chemiluminescence imaging system after developing the membranes with ECL western blotting reagent (Immobilon Western Chemiluminescent HRP Substrate, MERK-Millipore). The protein expressions of RUNX2 (60 kDa) and ALP (75 kDa) were quantified after normalization to GAPDH (37 kDa) expression and presented as the relative protein expression.

a and b of FIG. 15 and a and b of FIG. 16 show the results of examining stem cell adhesion while tuning the nanogaps by magnetic switching in the small nanoscreen (a and b of FIG. 15) and the large nanoscreen (a and b of FIG. 16). In a and b of FIG. 15 and a and b of FIG. 16, it can be seen that paxillin, actin, and nuclei decreased in the “low gap” and increased in the “high gap”, compared to the “medium gap”. However, in a of FIG. 15, it can be seen that the increase rates of paxillin, actin and nucleus in the “high gap” were significantly larger than those in the “low gap”. This difference can also be confirmed in b of FIG. 15 showing the results of quantifying the adhered cell density, cell size, focal adhesion number and aspect ratio. On the other hand, in a of FIG. 16, it can be seen that the decrease rates of paxillin, actin and nuclei in the “low gap” were significantly greater than those in the high gap. This difference can also be confirmed in b of FIG. 16 showing the results of quantifying the adhered cell density, cell size, focal adhesion number and aspect ratio. This means that, in the small nanoscreen, it is more effective to facilitate stem cell adhesion and differentiation by the “high gap”, and in the large nanoscreen, it is more effective to inhibit stem cell adhesion and differentiation by the “low gap”.

The above experimental results also mean that the direction in which a magnetic field is applied is important in regulating stem cell adhesion to the nanoscreen and stem cell differentiation on the nanoscreen. This is because the nanogap of the nanoscreen can form the “high gap” or the “low gap” depending on the direction in which the magnetic field is applied. To confirm this fact, the expression levels of various proteins were checked while tuning the nanogaps of the small nanoscreen and the large nanoscreen. a of FIG. 17 depicts images showing the expression levels of paxillin, actin/nuclei, integrin β1, and TAZ after culturing for 48 hours, and b of FIG. 17 depicts images showing the expression levels of RUNX2, actin/nuclei, and ALP after culturing for 5 days. In a and b of FIG. 17, it can be seen that the expression of the proteins is remarkably promoted when the small nanoscreen is in a “high gap” state, and is remarkably inhibited when the large nanoscreen is in a “low gap” state. This can also be confirmed in c of FIG. 17 showing the results of Western blotting of the amounts of RUNX2, ALP and GAPDH under each condition and can also be confirmed in graphs showing the results of relatively quantifying the protein expressions of RUNX2 and ALP. In addition, in a and b of FIG. 18, it can be seen that the expression pattern of YAP depending on changes the nanoscreen size and the nanogaps is the same as in a to c of FIG. 17. FIG. 19 depicts images and a quantification graph showing the expression pattern of osteocalcin, and as can be seen therein, the expression pattern is uniform depending on the direction of application of the magnetic field. FIG. 20 depicts graph showing the results of quantifying the adhered cell density, cell size, focal adherence number and aspect ratio, and shows results corresponding to the images and graph results shown in a to c of FIG. 17 and a and b of FIG. 18.

a and b of FIG. 21 show the results of testing whether paxillin and actin/nuclei are expressed even when a magnetic field is applied to a nanoscreen without a ligand. It can be seen that, even when the magnetic field was applied to each nanoscreen in the optimal direction, there was little change in the expression of paxillin and actin/nuclei compared to when no magnetic field was applied. In a and b of FIG. 21, N.S. signifies that there was no statistical difference between the compared groups.

### Experimental Example 5—Experiment on Regulation of Stem Cell Adhesion and Differentiation Using Nanoscreen In Vivo

As a proof-of-principle for in vivo translation of remotely controlling the nanogaps to regulate focal adhesion-dependent mechanotransduction of stem cells, nanoscreens were implanted into the subcutaneous pockets of nude mice. Two-month-old nude mice (40 mice) were subjected to surgery after obtaining the approval from the Institutional Animal Care and Use Committee of Korea University. Prior to the implantation of the nanoscreen, intraperitoneal injections of 15 μL each of zoletil and rompun were administered to the nude mice. 2 cm-long incisions were subsequently made on the backs of the mice, followed by suturing of the wound. Immediately after implantation, human mesenchymal stem cells (hMSCs) were subcutaneously injected onto the nanoscreen at a density of 300 k cells per nanoscreen. The focal adhesion and mechanotransduction of stem cells (hMSCs) were examined at 6 h post-injection after externally placing a magnet (300 mT) on the backs or abdomens of the mice to enable the magnetic switching of the magnetic screens in situ, or without placing the magnet adjacent to the mice. When the magnet is placed on the mouse back, the “high gap” is formed, and when the magnet is placed on the abdomen, the “low gap” is formed, and when the magnet is not placed, the “medium gap” is formed. This observation was performed throughout the entire post-implantation time of 6 hours. The implanted small nanoscreens (“medium gap” and “high gap”) or large nanoscreens (“medium gap” and “low gap”) were retrieved for immunofluorescence staining analysis of stem cell adhesion and mechanotransduction, including the identification of human cells by detecting human nuclear antigen (HuNu). In addition, SEM imaging analysis was performed to visualize and quantify the nanoscreens before and after implantation.

a and b of FIG. 22 depicts images and quantification graphs showing the expression of human nuclear antigen (HuNu), paxillin, actin/nuclei, and YAP after implanting the nanoscreens into the mice and applying the magnetic field. In all the images of HuNu in a of FIG. 22, it can be seen that HuNu became localized in the nuclei, suggesting that hMSCs preferentially adhered to the implanted nanoscreens. In the in vivo experimental results in a and b of FIG. 22, similar to the experimental results obtained in the laboratory, it can be seen that the adhered cell density, focal adhesion and protein expression were increased amplified when the small nanoscreen formed the “high gap”, and was inhibited when the large nanoscreen formed the “low gap”. As a result, it can be confirmed that, even when the nanoscreen is actually applied to a living organism, the adhesion and differentiation of stem cells can be regulated by tuning the nanoscreen through remote application of a magnetic field from outside the living organism.

In addition, a and b of FIG. 23 show test results indicating that the nanoscreen is stable in vivo and can be safely used. As shown in the figure, it can be seen that, at pre-implantation and 6 hours post-implantation) of the nanoscreen into the mouse, there was almost no change in the density of the magnetic screens bound to the substrate or the ligand-bearing GNP (GNP in the figure). In addition, it can be seen that there was almost no change in shape or size as well as density. This is the same even after the nanogap was tuned by applying the magnetic field after implanting the nanoscreen into the mice. This means that nano-magnetite, a material constituting the magnetic screens, is not toxic to living things and does not react in living things, and thus it can be used safely.

While the present invention has been described with reference to the particular illustrative embodiments, it will be understood by those skilled in the art to which the present invention pertains that the present invention may be embodied in other specific forms without departing from the technical spirit or essential characteristics of the present invention. Therefore, the embodiments described above are considered to be illustrative in all respects and not restrictive. Furthermore, the scope of the present invention is defined by the appended claims rather than the detailed description, and it should be understood that all modifications or variations derived from the meanings and scope of the present invention and equivalents thereto are included in the scope of the present invention.

What is claimed is:

1. A nanoscreen for regulating stem cell adhesion and differentiation comprising:
magnetic screens each comprising an aggregate of one or more magnetic particle units;
a linker connected to one side of each of the magnetic screens; and
a substrate connected to the magnetic screens via the linkers,
wherein the substrate comprises ligands to which stem cells adhere.

2. The nanoscreen of claim 1, wherein an average diameter of the magnetic screens includes any one or more of a first average diameter, a second average diameter, and a third average diameter,
wherein the first average diameter is 150 to 250 nm, the second average diameter is 450 to 580 nm, and the third average diameter is 650 to 900 nm.

3. The nanoscreen of claim 2, wherein a surface of each of the magnetic screens, which faces the substrate, is spaced apart from the ligand present on the substrate by a distance of a nanogap,
wherein the nanogap is reversibly changed by application of a magnetic field.

4. The nanoscreen of claim 3, wherein the average diameter of the magnetic screens includes the first average diameter, and the stem cell adhesion and differentiation is facilitated by elongating the linker and increasing the nanogap, through pulling of the magnetic screens in a direction away from the substrate by application of the magnetic field.

5. The nanoscreen of claim 3, wherein the average diameter of the magnetic screens includes the third average diameter, and the stem cell adhesion and differentiation is inhibited by compressing the linker and reducing the nanogap, through pulling of the magnetic screens in a direction toward the substrate by application of the magnetic field.

6. The nanoscreen of claim 1, wherein the linker comprises: a polyethylene glycol (PEG) portion; a first bonding portion which forms a chemical bond with the magnetic screen; and a second bonding portion which forms a chemical bond with the substrate.

7. The nanoscreen of claim 6, wherein the magnetic screen includes a carboxylate group (—COO⁻),
the first bonding portion comprises any one of an amino group (—NH₂) and a thiol group (—SH) and form a chemical bond with the carboxylate group of the magnetic screen, and
the second bonding portion comprises any one of a maleimide group and an alkenyl group (—C═C—) and forms a chemical bond with a thiol group (—SH) provided on the substrate.

8. The nanoscreen of claim 7, wherein the linker has a structure of the following Formula 1: 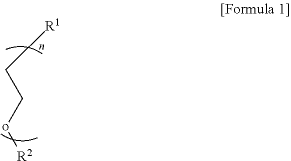
wherein n is 30 to 5,000, R¹ is any one of an amino group (—NH₂) and a thiol group (—SH), and R² is any one of a maleimide group and an alkenyl group (—C═C—).

9. The nanoscreen of claim 6, wherein the linker has a length of 10 nm to 1 μm.

10. The nanoscreen of claim 1, wherein the ligands provided on the substrate are bound to surfaces of gold nanoparticles bound to the substrate.

11. The nanoscreen of claim 10, wherein
the gold nanoparticles are provided on the substrate by chemical bonding with a portion of the thiol groups (—SH) provided on the substrate,
the ligands are bound to the gold nanoparticles, and
the linkers are connected to the substrate by chemical bonding with the other portion of the thiol groups (—SH) provided on the substrate.

12. The nanoscreen of claim 10, wherein the gold nanoparticles cover 0.001% to 10% of the area of the substrate.

13. The nanoscreen of claim 1, wherein 68 to 80% of the area of the substrate is covered by the magnetic screens.

14. The nanoscreen of claim 1, wherein the nanoscreen is prepared by:
forming aggregates of one or more magnetic particle units;
forming a carboxylate group on surfaces of the aggregates to form magnetic screens;
binding each of the magnetic screens to one end of each linker by stirring the magnetic screens and the linkers;
chemically binding the other end of each linker to thiol groups on a substrate on which thiol groups and ligands are present; and
deactivating thiol groups on the substrate, which remain unbound to the linkers.

15. The nanoscreen of claim 14, wherein the substrate comprises a glass substrate, and thiol group and ligands provided on at least one surface of the glass substrate,
the thiol groups are provided by thiolizing the glass substrate,
at least a portion of the thiol groups are bound to the gold nanoparticles, and
the ligands are bound to the gold nanoparticles bound to the thiol groups.

16. A method of regulating stem cell adhesion and differentiation using a nanoscreen, the method comprising regulating stem cell adhesion and differentiation by applying a magnetic field to the nanoscreen according to claim 1.

17. The method of claim 16, wherein the magnetic field is applied from outside the body to remotely control the nanoscreen in the body.

18. The method of claim 16, wherein the magnetic field has a strength of 100 mT to 500 mT.

19. The method of claim 16, wherein stem cell adhesion and differentiation is facilitated by elongating the linker through pulling of the magnetic screens in a direction away from the substrate by the magnetic field.

20. The method of claim 16, wherein stem cell adhesion and differentiation is inhibited by compressing the linker through pulling of the magnetic screens in a direction toward the substrate by the magnetic field.
